(19) United States
(12) Patent Application Publication
(10) Pub. No.: US 2019/0184047 A1
(43) Pub. Date: Jun. 20, 2019

(54) METHOD AND A DEVICE FOR STERILIZATION BY PLASMA

(71) Applicant: SOCIETE POUR LA CONCEPTION DES APPLICATIONS DES TECHNIQUES ELECTRONIQUES, Merignac (FR)

(72) Inventors: Sandrine VILLEGER, Montjoire (FR); Marie-Agnes BENOIT, Merignac (FR)

(21) Appl. No.: 15/879,910

(22) Filed: Jan. 25, 2018

(30) Foreign Application Priority Data
Dec. 20, 2017 (FR) 1762594

(51) Int. Cl. A61L 2/14; A61L 2/20

(52) U.S. Cl. CPC A61L 2/14; A61L 2/20; A61L 2202/122; A61L 2202/24; A61L 2202/11

(57) ABSTRACT

A method of sterilizing an object in a sterilization chamber comprises: evacuating the sterilization chamber at a pressure value; injecting a gaseous phase into at least one plasma generator via an ionization duct, the injected gaseous phase comprising dinitrogen with a volume content of dioxygen, a first ratio of the flow rate for injecting the gaseous phase in each plasma generator over the volume of the sterilization chamber being maintained at a value that is greater than or equal to 0.02 min⁻¹, and a second ratio of the flow rate for injecting the gaseous phase into each plasma generator over the inside sectional area of the ionization duct being maintained at a value that is less than or equal to 102 m/min; the at least one plasma generator generating a plasma from the gaseous phase; and injecting a post-discharge stream resulting from the plasma into the previously-evacuated sterilization chamber.

The present invention relates to a method and a device for sterilizing an object by using a post-discharge stream resulting from a plasma.

### BACKGROUND OF THE INVENTION

It is known to sterilize objects by means of an autoclave in which the object to be sterilized is raised to a determined high temperature, of the order of 120° C., and for this to be done during determined time periods with cycles that are specified by legislation.

However, sterilization by means of an autoclave may lead to certain objects being damaged, e.g. objects including polymer materials, due to a temperature that is too high.

Methods enabling sterilization to be performed at lower temperatures have consequently been developed in order to reduce the damage to objects while they are being treated.

In this context, devices have been proposed that make use of a post-discharge stream resulting from a plasma. The post-discharge from a plasma comprises neutral species resulting from the ions of the plasma that is formed by recombining with electrons. Those devices enable the object to be sterilized with the post-discharge stream resulting from the plasma produced by a plasma generator.

However, certain known techniques for sterilization by post-discharge stream are likely to generate ultraviolet (UV) radiation at very short wavelengths, which can damage the object to be sterilized.

In addition, in order to be able to sterilize more objects simultaneously or to sterilize larger objects, a need exists for sterilization devices having larger sterilization chambers, while maintaining the same quality of sterilization, and without excessively increasing the power rating of the plasma generator.

### OBJECT AND SUMMARY OF THE INVENTION

The invention aims to enable objects to be sterilized by using a post-discharge stream resulting from a plasma while reducing the risk of damaging the objects.

The invention also aims to increase the volume of the sterilization chamber, in particular so as to be able to sterilize more objects simultaneously, while ensuring proper sterilization and limiting the power required for the plasma generator.

In a first aspect, the invention provides a method of sterilizing an object, the method comprising:

placing the object inside a sterilization chamber;
evacuating the sterilization chamber containing the object down to a pressure value that is less than or equal to 0.3 millibars (mbar);
injecting a gaseous phase into at least one plasma generator via an ionization duct, the injected gaseous phase comprising dinitrogen with a volume content of dioxygen that is less than or equal to 1%, a first ratio R1 of the flow rate for injecting the gaseous phase in each plasma generator over the volume of the sterilization chamber being maintained at a value that is greater than or equal to 0.02 min⁻¹, and a second ratio R2 of the flow rate for injecting the gaseous phase into each plasma generator over the inside sectional area of the ionization duct being maintained at a value that is less than or equal to 102 meters per minute (m/min);
said at least one plasma generator generating a plasma from the gaseous phase; and
injecting a post-discharge stream resulting from the plasma into the previously-evacuated sterilization chamber containing the object via a plurality of injection orifices in communication with the ionization duct.

The inventors have found that the first flow rate ratio for injecting the gaseous phase into each plasma generator over the volume of the sterilization chamber should not be too small so as to be able to fill the sterilization chamber suitably with the post-discharge stream, and thus ensure proper sterilization of the objects situated inside the sterilization chamber.

The inventors have also found that the second ratio of the injection flow rate in each plasma generator over the section of the ionization duct should not be too great so as to ensure proper ionization of the gaseous phase by each plasma generator, and thus ensure proper sterilization of objects situated inside the sterilization chamber.

In addition, the residual oxygen inside the chamber is eliminated by lowering the pressure to 0.3 mbar before injecting the post-discharge stream, thus limiting the consumption of nitrogen atoms reacting with oxygen atoms, and also reducing the production of UV radiation inside the sterilization chamber as is produced by the reaction between the nitrogen atoms and the oxygen atoms.

Furthermore, the fact of ensuring dinitrogen purity in the gaseous phase such that the dioxygen content is less than 1% also makes it possible to reduce consumption of nitrogen atoms by residual oxygen atoms, and also reduces the production of UV radiation resulting from this reaction.

In addition, the fact that the post-discharge stream is injected into the sterilization chamber by a plurality of injection orifices makes it possible to obtain better uniformity of sterilizing species in the sterilization chamber, and thus makes it possible to ensure better sterilization for all of the objects present inside the sterilization chamber.

The method may also include the following characteristics, taken alone or in combination depending on technical possibilities:

the volume of the sterilization chamber divided by the number of injection orifices is less than or equal to 18 liters (L);
the gaseous phase is injected into a plurality of plasma generators;
each plasma generator is associated with a single injection orifice;
the first ratio R1 is maintained at a value that is less than or equal to 0.11 min⁻¹, and preferably less than or equal to 0.08 min⁻¹;
the first ratio R1 is maintained at a value that is greater than or equal to 0.04 min⁻¹;
the first ratio R1 is maintained in the range 0.04 min⁻¹ to 0.11 min⁻¹, and preferably in the range 0.04 min⁻¹ to 0.08 min⁻¹;
the second ratio R2 is maintained at greater than or equal to 20 m/min, and preferably at greater than or equal to 41 m/min;
the second ratio R2 is maintained at a value that is less than or equal to 77 m/min;
the second ratio R2 is maintained in the range 20 m/min to 77 m/min, and preferably in the range 41 m/min to 77 m/min;
the volume content of dioxygen in the injected gaseous phase is less than or equal to 0.5%;
the gaseous phase is obtained by filtering dioxygen from a stream of air; and
the object is a medical instrument.

In a second aspect, the invention provides a device for sterilizing an object, the device comprising:

a sterilization chamber defining a treatment zone in which the object to be sterilized is designed to be placed;
a gaseous phase source configured to deliver a gaseous phase including dinitrogen with a volume content that is less than or equal to 1%;
at least one plasma generator in communication with an ionization duct that is itself in communication with the gaseous phase source and that is configured to generate a plasma from the gaseous phase and to inject a post-discharge stream resulting from said plasma into the sterilization chamber; and
a pump configured to perform evacuation of the sterilization chamber;
wherein the sterilization device comprises a control system configured to control the pump so as to impose a pressure that is less than or equal to 0.3 mbar in the sterilization chamber before injection of the post-discharge stream, and to control injection of the gaseous phase into each plasma generator and maintaining a first ratio R1 of the flow rate for injecting the gaseous phase in each plasma generator over the volume of the sterilization chamber at a value that is greater than or equal to 0.02 min⁻¹, and by maintaining a second ratio R2 of the flow rate for injecting the gaseous phase in each plasma generator over the inside sectional area of the ionization duct at a value that is less than or equal to 102 m/min; and
wherein a plurality of injection orifices open out into said sterilization chamber and are in communication with the ionization duct.

### BRIEF DESCRIPTION OF THE DRAWING

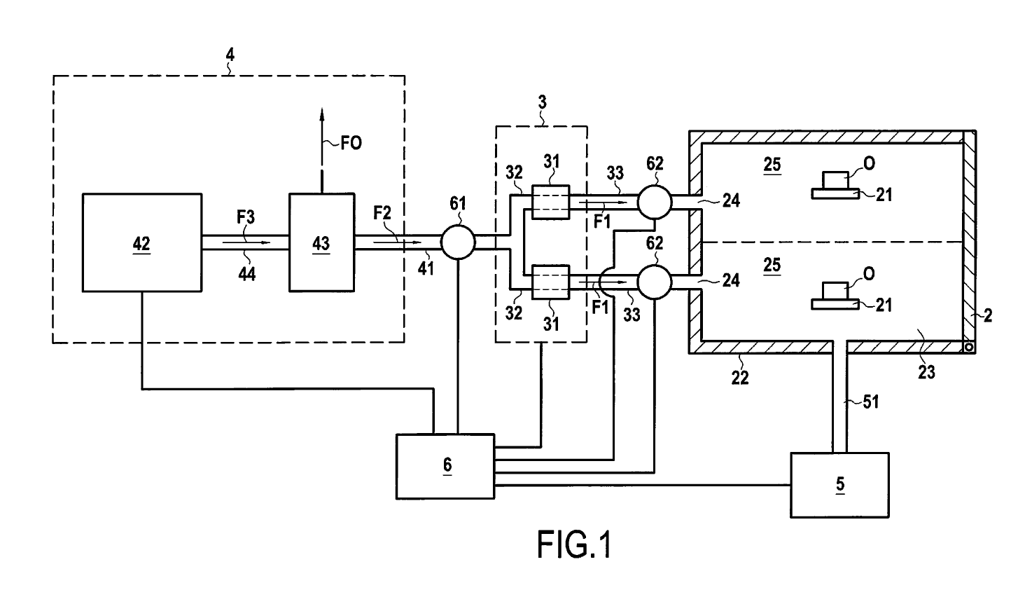

Other characteristics and advantages of the invention appear from the following description given in non-limiting manner and with reference to the accompanying FIG. 1, which is a diagram showing a sterilization device of the invention.

### DETAILED DESCRIPTION OF EMBODIMENTS

FIG. 1 is a diagram showing a sterilization device 1 configured to sterilize one or more objects O by treatment with a post-discharge stream resulting from a nitrogen plasma.

The objects O may be medical instruments, endoscopes, scissors, or scalpels. The invention is also advantageous for sterilizing objects other than medical instruments, such as electronic components, in particular electronic cards.

The objects O are placed in a sterilization chamber 2 having supports 21 on which said objects O are installed. The sterilization chamber 2 includes a wall 22 that defines an inside volume 23 in which the supports 21 and the objects O to be sterilized are situated. In this example, the supports 21 are situated one above another, so that the objects O are also situated one above another in the sterilization chamber 2.

In order to sterilize the objects O, a first post-discharge stream F1 resulting from a nitrogen plasma is injected into the sterilization chamber 2.

The device 1 comprises a plasma generation system 3 for generating a plasma so as to generate the post-discharge stream and inject it into the sterilization chamber 2. The plasma generation system 3 is connected to the sterilization chamber 2 by a plurality of injection ducts 33 that define injection orifices 24 formed in the wall 22 of the sterilization chamber 2 and via which the first post-discharge stream F1 penetrates into the inside volume 23 of said sterilization chamber 2. The fact that the post-discharge stream is injected into the sterilization chamber 2 by a plurality of injection orifices 24 makes it possible to better distribute said post-discharge stream in the sterilization chamber 2, and thus makes it possible to obtain more uniform injection of sterilizing nitrogen atoms into said sterilization chamber 2.

The plasma generation system 3 comprises a plurality of plasma generators 31 each of which is associated with an ionization duct 32 in which there flows a second stream F2 of a gaseous phase comprising dinitrogen. By way of example, the plasma generators 31 comprise magnetrons, which generate an electromagnetic field having waves that are directed towards the ionization duct 32 so as to ionize the gaseous phase. Each plasma generator 31 is associated with an injection duct 33 so as to inject the first post-discharge stream F1 into the sterilization chamber 2 through an injection orifice 24.

In the embodiment shown in FIG. 1, the plasma generation system 3 comprises two plasma generators 1. However, the plasma generation system 3 could comprise a single plasma generator 31, which in this example would be associated with a plurality of injection ducts 33 and with a plurality of injection orifices 24, or else it could have a larger number of plasma generators 31, which in this example would each be associated with a single injection duct 33 and with a single injection orifice 24. However, the fact of having a plurality of plasma generators 31 available, each associated with a respective ionization duct 32, for a given total flow rate of the first stream sent towards the sterilization chamber, provides the advantage of limiting heating in the ionization ducts 32. The plasma generation system 3 may in particular be made up of a plurality of plasma generators 31 having a given power rating, and having passing therethrough ionization ducts 32 of given section, in which a given flow rate of gaseous phase is injected.

In order to inject enough nitrogen atoms into the sterilization chamber 2, and thus ensure proper sterilization of the objects O, a first ratio R1 of the flow rate for injecting the gaseous phase in each plasma generator 31 (in other words the flow rate in the ionization ducts 32) over the volume of the sterilization chamber 2 is maintained at a value that is greater than or equal to 0.02 min⁻¹. A first ratio R1 that is less than 0.02 min⁻¹ tends to lengthen the time required to sterilize the objects O excessively. In advantageous manner, the first ratio R1 is maintained at a value that is less than or equal to 0.11 min⁻¹, in such a manner as to limit generating UV radiation. Preferably, in order to further improve filling of the tank and uniformity of the post-discharge stream inside the sterilization chamber 2, the first ratio R1 is greater than or equal to 0.04 min⁻¹. In preferred manner, in order to even further limit the production of UV radiation, the first ratio R1 is less than or equal to 0.08 min⁻¹. The inventors have found that it is preferable not to increase the flow rate of nitrogen atoms arriving in the sterilization chamber 2 too greatly, since that increases the production of UV radiation as a result of nitrogen atoms combining with the residual oxygen atoms that are present. Despite the quantity of oxygen atoms being small, the increase in the quantity of nitrogen atoms increases the probability of a reaction between nitrogen and oxygen atoms, which reaction produces UV radiation.

In addition, in order to obtain good ionization of the gaseous phase, and thus produce a quantity of nitrogen atoms that is sufficient for sterilizing the objects O, a second ratio R2 of the flow rate for injecting the gaseous phase in each plasma generator 31 (in other words the flow rate in the ionization ducts 32) over the inside sectional area of the ionization duct 32 is maintained at a value that is less than or equal to 102 m/min. The inside sectional area of an ionization duct 32 corresponds to the area, taken perpendicularly relative to the length of the ionization duct 32, defined by the inside wall of said duct 32. In advantageous manner, the second ratio R2 is maintained at a value that is greater than or equal to 20 m/min, so that the plasma generation system 3 generates more nitrogen atoms in order to improve sterilization of the objects O. The second ratio R2 is preferably less than or equal to 77 m/min.

As shown in FIG. 1, the injection orifices 24 are distributed along the length of the sterilization chamber 2 in such a manner as to divide the inside volume 23 of said sterilization chamber into the same number of elementary volumes 25, each associated with a respective injection orifice 24. The number of injection orifices 24 is adapted so that each elementary volume 25 holds 18 L at most. In other words, the volume of the sterilization chamber 2 divided by the number of injection orifices 24 is less than or equal to 18 L. An elementary volume less than or equal to 18 L makes it possible to ensure a concentration of nitrogen atoms in each elementary volume 25 that makes it possible to improve sterilization of the objects O.

In a variant not shown, the injection orifices 24 are situated in at least two opposite walls of the sterilization chamber 2, which tends to even further improve uniformity of distribution of the first post-discharge stream F1 inside said sterilization chamber 2. According to another possible characteristic, for each injection orifice 24 situated in a first wall of the injection chamber 2, said injection chamber 2 includes another injection orifice 24 situated in a second wall opposite the first wall.

In order to feed the plasma generation system 3 with the second stream F2 of gaseous phase comprising nitrogen, the device 1 comprises a gaseous phase source 4 that is connected to the plasma generation system by a feed duct 41.

In the embodiment shown in FIG. 1, the gaseous phase source 4 generates the second stream F2 by filtering dioxygen from a third stream F3 of air. To do this, the gaseous phase source 4 comprises a source 42 of air, e.g. a compressor or a cylinder of compressed air, which is connected to a filter 43 via a duct 44 in which the third stream of air F3 flows. The filter 43 comprises a membrane in which the third stream of air F3 flows and that is configured to separate the third stream F3 of air firstly into a stream of dioxygen FO, and secondly into a stream constituted mainly of dinitrogen forming the second stream F2. The filter 43 may for example be a filter of the “HiFluxx TT604®” type sold by “Parker®”. In another possible embodiment, the gaseous phase source 4 may be formed by at least one tank of dinitrogen.

The gaseous phase source 4 is configured so that the gaseous phase includes a dioxygen volume content that is less than or equal to 1%, and preferably less than or equal to 0.5%, so as firstly to limit UV production, and secondly to maximize the number of nitrogen atoms for sterilizing objects O by limiting the consumption of nitrogen atoms for combining with oxygen. By way of example, this may be achieved by selecting a suitable filter 41, or by using a tank of dinitrogen as a gaseous phase source with a volume content that is appropriate.

In addition, the device 1 also comprises a pump 5 that is connected via an evacuation duct 51 to the sterilization chamber 2 and that is configured to reduce the pressure inside said sterilization chamber 2 and thus to perform evacuation. The pump 5 is configured to establish a pressure that is less than 0.3 mbar inside the sterilization chamber 2 before injecting the gaseous phase in such a manner as to eliminate residual oxygen inside said sterilization chamber 2, and thus firstly limit UV production, and secondly optimize sterilization by limiting the combination of nitrogen atoms with the oxygen, these recombined nitrogen atoms no longer performing the function of sterilizing objects O.

The device 1 further comprises a control system 6 that is connected to the pump 5, to the plasma generation system 3, and to the gaseous phase source 4, and more particularly to the air source 42 when the gaseous phase is obtained by filtering air. The control system 6 is also connected to a flow rate sensor 61 situated on the feed duct 41 and that is configured to measure the flow rate of the second stream F2 injected into the plasma generation system 3 and to transmit this measurement to the control system 6. The control system 6 may also be connected to flow rate sensors 62 that are situated on each of the injection ducts 33 and that are configured to measure the flow rate of the first stream F1 in each injection duct 33 and to transmit the measurement to the control system 6. The control system 6 comprises a processor and a memory in which a computer program is stored for implementing the sterilization method of the invention.

The control system 6 is configured to control the pump 5 and to evacuate the sterilization chamber 2 once the objects O have been placed on the supports 21, thus establishing a pressure that is less than or equal to 0.3 mbar inside said sterilization chamber 2. Once evacuation has been performed, the control system 6 is configured to control the production of plasma by controlling the gaseous phase source 4 and the plasma generation system 3, and to maintain the first ratio R1 (flow rate for injecting the gaseous phase in each plasma generator 31 over the volume of the sterilization chamber 2) at a value that is greater than or equal to 0.02 min⁻¹, and to maintain the second ratio R2 (flow rate for injecting the gaseous phase in each plasma generator 31 over the inside sectional area of the ionization duct 32) at a value that is less than or equal to 102 m/min. As a function of the flow rate measurements of the sensors 61 and 62, the control system 6 controls the flow rate of the gaseous phase injected by the gaseous phase source 4 so as to maintain the first ratio R1 and the second ratio R2 at the desired values. The post-discharge stream resulting from the plasma is injected into the sterilization chamber 2 via each of the injection orifices 24.

In an example of a possible embodiment, the sterilization chamber 2 has a sterilization volume of 19.5 L, and includes four injection orifices 24 that divide said inside volume of the sterilization chamber into four elementary volumes 25 of 8.25 L, each elementary volume 25 being associated with a respective injection orifice 24. The injection orifices 24 are divided into two groups of two injection orifices 24, the two groups being situated on opposite faces of the sterilization chamber 2. The plasma generation system 3 comprises four plasma generators 31 each having a magnetron with a power rating of 300 Watts (W), each of which is associated with a respective injection orifice 24. A higher power rating for the magnetrons could be used, e.g. 600 W. However, it is preferable for the power rating at which the magnetrons are used not to be greater than 600 W so as to limit heating in the ionization ducts. The ionization ducts 32 of the plasma generators 31 all have an inside diameter of 5 millimeters (mm), and therefore a section of 19.6 square millimeters (mm²).

Table 1 below shows the results of tests that have been carried out by injecting into each ionization duct 32 a gaseous phase of dinitrogen, with a volume content of dinitrogen of less than 1%, for seven different flow rate values. For each test, the sterilization chamber 2 was evacuated before the injection by reducing its pressure to a value of 0.3 mbar.

TABLE 1

| | | | chamber | | | | R2 |
|---|---|---|---|---|---|---|---|
| | flow rate per | total flow rate | pressure during | | | R1 | (m/ |
| | duct | of chamber | injection | √IN2* | INOβ | (min⁻¹) | min) |
| test 1 | 0.4 L/min | 1.6 L/min | 5 mbar | 38.73 | 1500 | 0.020 | 20 |
| test 2 | 0.8 L/min | 2.4 L/min | 8 mbar | 44.72 | 3500 | 0.041 | 41 |
| test 3 | 1 L/min | 4 L/min | 9 mbar | 50 | 4000 | 0.051 | 51 |
| test 4 | 1.5 L/min | 6 L/min | 10 mbar | 50 | 5500 | 0.077 | 77 |
| test 5 | 1.6 L/min | 6.4 L/min | 12 mbar | 54.77 | 7000 | 0.082 | 82 |
| test 6 | 1.8 L/min | 7.2 L/min | 16 mbar | 59.16 | 8000 | 0.092 | 92 |
| test 7 | 2 L/min | 8 L/min | 18 mbar | 63 | 8000 | 0.103 | 102 |

The tests that were performed, with a flow rate in each ionization duct varying from 0.4 liters per minute (L/min) to 2 L/min, were satisfactory since the quantity of nitrogen atoms produced are sufficient for sterilizing the objects O, and the quantity of UV radiation produced was not excessive, thereby limiting any risk of damaging said objects O. In the tests that were performed, the strength of the UV radiation (INOβ) appears to have remained unchanged from test 6 to test 7. However, this lack of change was caused by the optical fiber sensor used for measuring the strength of UV radiation, since its sensitivity is affected by the disturbances in the jet of the post-discharge stream, which disturbances increase with flow rate. UV radiation did indeed increase from test 6 to test 7.

In another example of a possible embodiment, the sterilization chamber 2 has a sterilization volume of 141.7 L, and included eight injection orifices 24 that divided said inside volume of the sterilization chamber into eight elementary volumes of 17.7 L, each elementary volume being associated with a respective injection orifice 24. The injection orifices 24 were distributed in two groups of four injection orifices 24, the two groups being situated on opposite faces of the sterilization chamber 2. The plasma generation system 3 had eight plasma generators 31, each having a magnetron with a power rating of 300 W, and each was associated with a respective injection orifice 24. The ionization ducts 32 of the plasma generators 31 all had an inside diameter of 5 mm, and therefore a section of 19.6 mm².

1. A method of sterilizing an object, the method comprising:
placing the object inside a sterilization chamber;
evacuating the sterilization chamber containing the object down to a pressure value that is less than or equal to 0.3 mbar;
injecting a gaseous phase into at least one plasma generator via an ionization duct, the injected gaseous phase comprising dinitrogen with a volume content of dioxygen that is less than or equal to 1%, a first ratio R1 of the flow rate for injecting the gaseous phase in each plasma generator over the volume of the sterilization chamber being maintained at a value that is greater than or equal to 0.02 min⁻¹, and a second ratio R2 of the flow rate for injecting the gaseous phase into each plasma generator over the inside sectional area of the ionization duct being maintained at a value that is less than or equal to 102 m/min;
said at least one plasma generator generating a plasma from the gaseous phase; and
injecting a post-discharge stream resulting from the plasma into the previously-evacuated sterilization chamber containing the object via a plurality of injection orifices in communication with the ionization duct.

2. A method according to claim 1, wherein the first ratio R1 is maintained at a value that is less than or equal to 0.11 min⁻¹.

3. A method according to claim 2, wherein the first ratio R1 is maintained at a value that is less than or equal to 0.08 min⁻¹.

4. A method according to claim 1, wherein the first ratio R1 is maintained at a value that is greater than or equal to 0.04 min⁻¹.

5. A method according to claim 1, wherein the second ratio R2 is maintained at a value that is greater than or equal to 20 m/min.

6. A method according to claim 5, wherein the second ratio R2 is maintained at a value that is greater than or equal to 41 m/min.

7. A method according to claim 1, wherein the second ratio R2 is maintained at a value that is less than or equal to 77 m/min.

8. A method according to claim 1, wherein the gaseous phase is injected into a plurality of plasma generators.

9. A method according to claim 1, wherein the volume of the sterilization chamber divided by the number of injection orifices is less than or equal to 18 L.

10. A method according to claim 1, wherein the volume content of dioxygen in the injected gaseous phase is less than or equal to 0.5%.

11. A method according to claim 1, wherein the gaseous phase is obtained by filtering dioxygen from a stream of air.

12. A method according to claim 1, wherein the object is a medical instrument.

13. A device for sterilizing an object, the device comprising:
a sterilization chamber defining a treatment zone in which the object to be sterilized is designed to be placed;
a gaseous phase source configured to deliver a gaseous phase including dinitrogen with a volume content that is less than or equal to 1%;
at least one plasma generator in communication with an ionization duct that is itself in communication with the gaseous phase source and that is configured to generate a plasma from the gaseous phase and to inject a post-discharge stream resulting from said plasma into the sterilization chamber; and
a pump configured to perform evacuation of the sterilization chamber;
wherein the sterilization device comprises a control system configured to control the pump so as to impose a pressure that is less than or equal to 0.3 mbar in the sterilization chamber before injection of the post-discharge stream, and to control injection of the gaseous phase into each plasma generator and maintaining a first ratio R1 of the flow rate for injecting the gaseous phase in each plasma generator over the volume of the sterilization chamber at a value that is greater than or equal to 0.02 min⁻¹, and by maintaining a second ratio R2 of the flow rate for injecting the gaseous phase in each plasma generator over the inside sectional area of the ionization duct at a value that is less than or equal to 102 m/min; and
wherein a plurality of injection orifices open out into said sterilization chamber and are in communication with the ionization duct.
